(19) United States
(12) Patent Application Publication
(10) Pub. No.: US 2025/0229027 A1
(43) Pub. Date: Jul. 17, 2025

(54) CARTRIDGE ASSEMBLY AND FIXING MEMBER

(71) Applicant: Sanofi, Paris (FR)

(72) Inventors: Michael Helmer, Frankfurt am Main (DE); Reinhard Rieger, Obertshausen (DE); Martin Vitt, Frankfurt am Main (DE)

(21) Appl. No.: 18/847,588

(22) PCT Filed: Mar. 15, 2023
(86) PCT No.: PCT/EP2023/056534

§ 371 Date: Sep. 16, 2024

(30) Foreign Application Priority Data
Mar. 17, 2022 (EP) 22315067.3

(51) Int. Cl. A61M 5/24

(52) U.S. Cl. CPC A61M 5/2422; A61M 2005/2437; A61M 2005/244; A61M 2207/00

(57) ABSTRACT

The present disclosure relates to a cartridge assembly provided with a fixing member, wherein the fixing member is configured for fastening to a cartridge, wherein the cartridge is configured for insertion into a housing component of a drug delivery device, wherein the housing component comprises a sidewall confining a cartridge receiving space, the cartridge receiving space extending in a longitudinal direction (z), the fixing member comprising:
a fastening portion for fastening to a neck portion of the cartridge,
a fixing structure comprising a first fixing element configured to engage with a counter fixing structure of the housing component, and
an alignment portion configured to align with a counter alignment structure provided on or in the sidewall of the cartridge holder.

### CROSS-REFERENCE TO RELATED APPLICATIONS

This is a National Stage Application, filed under 35 U.S.C. § 371, of International Patent Application No. PCT/EP2023/056534, filed on Mar. 15, 2023, which claims priority to European Patent Application No. 22315067.3, filed on Mar. 17, 2022, the disclosures of which are incorporated herein by reference in their entireties.

### TECHNICAL FIELD

The present disclosure relates to a cartridge assembly, particularly a cartridge assembly for a drug delivery device, such as an injection device and/or a pen-type injector. The present disclosure further relates to a fixing member for use in a cartridge assembly, to a drug delivery device and to a method of assembling a cartridge assembly.

### Background

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of variable size. Moreover, a dose setting as well as a dose dispensing procedure must be easy to operate and has to be unambiguous.

A patient suffering from a particular disease may require a certain amount of a medicament to either be injected via a pen-type injection syringe or infused via a pump. With respect to reusable injection or delivery devices, a patient may have to load or to replace a cartridge. Reusable injection devices typically comprise a multi-component housing. For instance, the housing may comprise a proximal housing component, such as a body and a distal housing component, such as a cartridge holder detachably connectable to the body. Once a medicament provided in a medicament container, such as a cartridge, is empty, the cartridge holder may be disconnected from the body of the injection device and the empty cartridge may be removed and replaced with a new cartridge.

Another concern that may arise from cartridges being manufactured in essentially standard sizes and manufactured to comply with certain recognized local and international standards. Consequently, such cartridges are typically supplied in standard sized cartridges (e.g. 3 ml cartridges). Therefore, there may be a variety of cartridges supplied by a number of different suppliers and containing a different medicament but fitting a single drug delivery device. As just one example, a first cartridge containing a first medicament from a first supplier may fit a drug delivery device provided by a second supplier. As such, a user might be able to load an incorrect cartridge or medicament into a drug delivery device and, then, dispense said medicament (such as a rapid or basal type of insulin) without being aware that the medical delivery device was perhaps neither designed nor intended to be used with such a cartridge.

As such, there is a growing desire from users, health care providers, caregivers, regulatory entities, and medical device suppliers to reduce the potential risk of a user loading an incorrect drug type into a drug delivery device. It is also desirable to reduce the risk of dispensing an incorrect medicament (or the wrong concentration of the medicament) from such a drug delivery device.

Some approaches to avoid unintended cross use of cartridges with non-intended drug delivery devices include a mechanical fixing of a cartridge to a housing component of the drug delivery device, such as a cartridge holder. The cartridge holder in turn may be then detachably or non-detachably connectable to a dose mechanism of such drug delivery devices.

Cartridges configured for insertion into a cartridge holder of a drug delivery device typically comprises a tubular-shaped main body merging into a radially narrowed neck, which in turn merges into a distally located and radially widened head portion. The head portion is typically covered by a septum retainer, e.g. made of a rather pliable material, such as an aluminum cap. The septum retainer is configured to fasten a pierceable septum at a distal end of the head portion. With some approaches for fixing a cartridge in a cartridge holder the cartridge holder comprises a radially inwardly protruding structure, which is configured to engage the radially narrowed neck portion and/or to grip under the beaded cap of the cartridge. With such approaches and upon insertion of the cartridge into the cartridge holder the septum retainer may collide with a protruding structure of the cartridge holder, which may then lead to mechanical and/or visual defects in the region of the septum retainer of the cartridge.

It is therefore desirable to provide an improved cartridge assembly, which provides a reliable and durable fixing of the cartridge in a cartridge holder and which avoids damages or defects of the cartridge upon insertion of the cartridge into the cartridge holder.

### SUMMARY

In one aspect there is provided a fixing member for fastening to a cartridge. The cartridge is configured for insertion into a housing component of a drug delivery device, e.g. of an injection device, such as a pen type injector. The housing component of the drug delivery device comprises a sidewall confining a cartridge receiving space. The cartridge receiving space extends in a longitudinal direction, e.g. denoted as a longitudinal axial direction (z). The fixing member comprises a fastening portion for fastening to a neck portion of the cartridge. The fixing member further comprises a fixing structure. The fixing structure comprises at least a first fixing element configured to engage with a counter fixing structure of the housing component. The fixing member further comprises an alignment portion configured to align with a counter alignment structure provided on or in the sidewall of the housing component.

Typically, the fastening portion provides a fastening of the fixing member to the neck portion of the cartridge. By way of the fastening portion the fixing member can be fixed to the cartridge. The fastening portion typically comprises a mechanical fastening feature to engage with the neck portion of the cartridge. With some examples the fastening portion comprises a fastening clip for clip-fastening to the neck portion of the cartridge.

The fixing structure is configured to engage with the complementary shaped counter fixing structure of the housing component. By way of the fixing structure, the fixing member can be fixed, fastened and/or retained to the housing component. With some examples, the fixing member serves to fix the fixing member to the housing at least with regard to the longitudinal direction.

Typically, the counter fixing structure is provided on the sidewall confining the cartridge receiving space. The fixing structure may be provided on an outside facing portion of the fixing member and may face towards an inside of the sidewall of the housing component when the fixing member fastened to the cartridge is accommodated in the cartridge receiving space together with the cartridge.

The alignment portion is configured to align with a complementary shaped counter alignment structure provided on or in the sidewall. The counter alignment structure is typically provided on an inside of the sidewall confining the cartridge receiving space.

With some examples, the alignment portion helps and/or is configured to orient the fixing member in at least one correct or dedicated orientation relative to the housing component or housing portion and/or relative to the counter fixing structure. In this way the alignment portion helps to correctly align or to correctly orient the fixing member relative to the sidewall and hence relative to the counter fixing structure as provided on or in the sidewall. In this way, and by correctly aligning the alignment portion of the fixing member with the counter alignment structure of the sidewall the fixing structure of the fixing member can be appropriately aligned or oriented with regard to the counter fixing structure as provided on or in the housing component, e.g. on or in the sidewall of the housing component. Consequently, and when correctly aligned, the fixing member attached or fastened to the cartridge can be inserted together with the cartridge in longitudinal direction into the cartridge receiving space until the cartridge and/or the fixing member reaches a final assembly position inside the cartridge receiving space.

Upon reaching the final assembly position the fastening portion of the fixing member is correctly aligned with the counter fixing structure due to the mutual engagement of the alignment portion with the counter alignment structure. When reaching the final assembly configuration the fixing structure engages the counter fixing structure and serves to fix the fixing member and hence the cartridge connected thereto to the housing component at least with regards to the longitudinal direction.

With some examples the fixing member is attachable to the neck portion of the cartridge, which neck portion is provided at or near a distal end of the cartridge. The counter fixing structure of the housing component may be also provided at or near a distal end of the housing component and/or at or near a distal end of the cartridge receiving space. The cartridge provided with the fixing member may have to be inserted into the cartridge receiving space through an insert opening, which may be provided at or near a proximal end of the housing component. A correct mechanical engagement of the fixing structure with the counter fixing structure may require a correct alignment and/or correct orientation of the fixing structure relative to the counter fixing structure as seen in transverse direction, e.g. as seen with regards to an axis of rotation e.g. coinciding with the longitudinal direction.

Hence, the fixing structure may only correctly engage with the complementary shaped counter fixing structure when correctly oriented or aligned with regards to an axis of rotation extending along the longitudinal direction.

With some examples, the counter alignment structure extends along the longitudinal direction. The alignment portion of the fixing member is configured to engage and/or to align with the counter alignment structure, which counter alignment structure may extend from a proximal end towards a distal end of the cartridge receiving space. The counter alignment structure may comprise a rather straight shape and may extend along the longitudinal direction of the sidewall confining the cartridge receiving space. By way of the counter alignment structure as provided on or in the sidewall of the cartridge receiving space the fixing member can be correctly aligned and oriented with regards to an axis of rotation.

With some examples inserting of the fixing member into the housing component when fastened to the neck portion of the cartridge may be only allowed and may be only possible with the alignment portion correctly aligned or correctly oriented with regard to the counter alignment structure. There may be provided two, three or even more allowable or supported alignment configurations between the alignment portion of the fixing member and the counter alignment structure of the sidewall of the housing. With all available or conceivable mutual orientations or alignments of the alignment portion with the counter alignment structure the fixing structure will be able to engage the counter fixing structure.

With some examples and with all other orientations or alignments of the alignment portion relative to the counter alignment structure, i.e. when the alignment portion is misaligned relative to the counter alignment structure, the fixing structure will not be able to engage the counter fixing structure of the housing component. With these other and non-supported alignments insertion of the cartridge or fixing member into the cartridge receiving space may be blocked and/or prevented. Here, it may be of particular benefit that the alignment portion prevents insertion of the cartridge together with the fixing member into the cartridge receiving space as long as it is improperly aligned with regards to the counter alignment structure.

In this way, the counter alignment structure and the alignment portion provide a kind of a mechanical coding between the fixing member and the cartridge receiving space. In a non-matching configuration, in which the alignment portion is e.g. incorrectly aligned or incorrectly oriented with regards to the counter alignment structure the alignment portion blocks an insertion of the fixing member and the cartridge into the cartridge receiving space, e.g. by a proximally located insert opening of the housing component. In an allowable or supported configuration, in which the alignment portion of the fixing member is correctly aligned or oriented with the counter alignment structure the alignment portion is configured to slide along the counter alignment structure as the fixing member fixed to the cartridge is pushed into the cartridge receiving space into the insert opening until the cartridge and/or the fixing member reach a final assembly position.

When correctly aligned or oriented as defined by the mutual engagement of the alignment portion with the counter alignment structure the fixing structure of the fixing member is configured to correctly engage with the complementary shaped counter fixing structure of the housing component, thereby fixing the fixing member and hence the cartridge fixed or fastened thereto to the housing component at least with regards to the longitudinal direction (z). In effect, and by way of the fixing structure engaged with the complementary shaped counter fixing structure the fixing member and hence the cartridge connected thereto can be secured against a proximally directed removal from the cartridge receiving space.

According to a further example the fixing structure of the fixing member is configured to engage with the counter fixing structure in longitudinal direction and to fix the cartridge relative to the housing component with regard to the longitudinal direction. The fixing structure may be configured or implemented as a form fitting fixing structure. It may comprise a snap element to engage with a complementary shaped counter snap element of the counter fixing structure. The fixing structure and the counter fixing structure may be engageable by providing the fixing structure relative to the counter fixing structure in longitudinal distal direction. When reaching the final assembly configuration complementary shaped stop faces of the fixing structure and the counter fixing structure may engage thereby preventing a movement of the fixing structure in the opposite direction, hence preventing a movement of the fixing structure towards the proximal direction relative to the counter fixing structure.

The fixing structure comprises at least one of a radial protrusion and a radial recess to engage with one of a radial recess and a radial protrusion of the counter fixing structure. The radial protrusion may be provided with a beveled edge so as to induce a radial deformation in the course of approaching a final assembly configuration or upon reaching the final assembly configuration. In the final assembly configuration complementary shaped stop faces of the fixing structure and the counter fixing structure may engage and may abut in longitudinal direction, thereby retaining the fixing structure and hence the fixing member with the cartridge attached thereto inside the cartridge receiving space.

According to a further example the alignment portion comprises one of a radial protrusion and a radial alignment recess to engage with one of a complementary shaped radial alignment recess and a radial protrusion as provided on an inside surface of the sidewall of the housing component. With some examples the alignment portion of the fixing member comprises a radially outwardly extending protrusion to engage with a complementary shaped radial alignment recess as provided on the inside of the sidewall of the housing component. The radial alignment recess may comprise a longitudinal groove effectively extending all along the cartridge receiving space. The radial alignment recess may merge into the insert opening so as to define at least one or several allowable alignment or orientation configurations of the fixing member relative to the sidewall of the housing.

When correctly aligned and hence when the radial protrusion of the alignment portion engages the complementary shaped radial alignment recess of the sidewall of the housing component an insertion of the fixing member and the cartridge attached thereto into the cartridge receiving space is allowed and supported. As long as the radial protrusion is out of alignment with the complementary shaped radial alignment recess a respective insert motion into the cartridge receiving space is effectively blocked. Then, the radial protrusion may abut with a proximal end face of the sidewall confining the cartridge receiving space, thereby inhibiting an insertion of the cartridge and the fixing member into the cartridge receiving space.

With further examples the alignment portion of the fixing member comprises a radial alignment recess on its outside surface to engage with a complementary shaped radial protrusion protruding radially inwardly from an inside surface of the sidewall of the housing component. In this way there can be provided a likewise rotational alignment or orientation for inserting the fixing member and the cartridge attached thereto into the cartridge receiving recess.

With either example of a radial protrusion provided on one of the alignment portion and the inside of the sidewall of the housing component the mutual engagement of the radial protrusion with the radial alignment recess on the other one of the alignment portion and the inside of the sidewall of the housing component thee is provided a rotation inhibiting engagement or a keyed engagement between the fixing member and the cartridge receiving space. In either way, the counter alignment structure as provided on or in the sidewall of the housing component provides a kind of a symmetry breaking feature thus defining at least one or several predefined orientations or alignments of the fixing member relative to the housing component or cartridge receiving space.

In other words, a mechanical coding as provided by the alignment portion of the fixing member and the complementary shaped counter alignment structure of the sidewall limit the number of possible orientations of the fixing member relative to the cartridge receiving space. This way there will be only a limited number of configurations, in which the fixing structure is engageable with the counter fixing structure. In this way, the mutual engagement of the alignment portion of the fixing member with the counter alignment structure on or in the sidewall of the housing component provides a twofold function. First of all, it prevents a rotation of the fixing member relative to the housing component upon inserting the cartridge into the housing component. Second, it provides and enables a guiding function for that the fixing structure as provided on the fixing member intentionally engages the complementary shaped counter fixing structure on the inside of the housing component, which is may not be accessible from outside the housing component.

According to a further example the fastening portion comprises a fastening ring. The fastening ring comprises a first ring portion and a second ring portion. The first ring portion and the second ring portion are separated from each other by a slit opening. Insofar the fastening ring is and interrupted ring intersected by the slit opening thus forming a through opening through the fastening ring. Apart from the slit opening the fastening ring may be of annular shape. The slit opening provides an increased degree elasticity of the first and second ring portions. First and second ring portions may be of a somewhat semi-circular shape. That portions or sections of the first and second ring portions facing away from the slit opening may be mutually connected.

With some examples the fixing member and/or the ring portion of the fastening portion is unitarily formed. It is made of a single piece. The fixing member may comprise a unitary body. The unitary body may comprise a plastic component, e.g. an injection molded plastic component. With other examples the fixing member and/or if fastening ring may comprise a metal component. Also here the fastening ring and/or the body of the fixing member may comprise a single piece.

The fastening ring with first and second ring portions is suitable for fastening to the neck portion of the cartridge. The cartridge typically comprises a tubular-shaped barrel, extending into a radially narrowed neck portion via a radially narrowing shoulder. The neck portion may further extend into a slightly radially widened head portion. The head portion is typically provided with a seal, e.g. a pierceable septum.

The first and second ring portions separated from each other by a slit opening are elastically deformation relative to each other, thus allowing for a clip fastening onto the neck portion of the cartridge. The slit opening may be slightly smaller than a diameter of the neck portion of the cartridge. It may be brought in radial engagement with the neck portion of the cartridge. By applying a radially inwardly directed pressure onto the fastening ring, the first and second ring portions may become subject to a slight radially outwardly directed deformation, thereby temporarily increasing the size of the slit opening so as to receive the neck portion inside the aperture as formed by the fastening ring. After or when receiving the neck portion of the cartridge between the first and second ring portions the first and second ring portions can relax towards an initial, e.g. unbiased state, in which the first and second ring portions tightly embrace the neck portion. In this way, the first and second ring portions engage the neck portion in a form fitting manner. In addition, first and second ring portion may even get into a friction fit with the neck portion of the cartridge. By way of a friction fit the fastening ring and hence the fastening portion of the fixing member may be even secured against rotation relative to the cartridge.

According to a further example the fastening ring comprises a middle portion interconnecting the first ring portion and the second ring portion. The first and the second ring portions may directly adjoin or merge into each other. Then, the merging section may be denoted as the middle portion. The middle portion may be structurally enhanced compared to the first and second ring portions, thereby increasing the stability of the fastening portion.

According to a further example the alignment portion is provided on an outside surface of the middle portion. The alignment portion may be directly connected to the fastening portion. It may comprise a longitudinal extension connected to or unitarily formed with the fastening portion. Typically, the fastening portion, the fixing structure and the alignment portion are substantially fixed relative to each other. Apart from an intrinsic elastic deformation capability they are expediently immobile relative to each other.

By providing the alignment portion on an outside surface of the middle portion the alignment portion may break the annular symmetry of the fastening ring. In addition, any forces present to the alignment portion may be equally distributed to the first and second ring portions protruding from the alignment portion in opposite circumferential direction or tangential direction.

With a further example the fixing structure comprises a second fixing element. Here, the first fixing element is provided on the first ring portion. The second fixing element is provided on the second ring portion. With some examples the first fixing element and the second fixing element may be provided at diametrically opposite positions on the fastening ring. This way, any mechanical forces to be transferred via the first and second fixing elements can be somewhat symmetrically transferred into the fastening ring. By way of a first fixing element and a second fixing element there can be also provided a twofold mechanical fixing between the fixing structure and the complementary shaped counter fixing structure.

In this way, the stability of the mechanical fastening as provided by the fixing member for keeping the cartridge inside the cartridge receiving space can be improved. In addition, and by having first and second fixing elements on opposite sides of the fastening ring there can be provided a redundant mechanical fixing of the fixing member and hence of the cartridge to the cartridge receiving space and finally to the housing component of the drug delivery device.

With some examples the first fixing element and/or the second fixing element comprise a radially outwardly extending protrusion to engage with a complementary shaped recess as provided by the counter fixing structure. In this way, the counter fixing structure can be void of radially inwardly protruding features or structures that could otherwise harm the integrity of the cartridge or of its septum retainer.

According to a further example the alignment portion of the fixing member comprises a longitudinal extension protruding in longitudinal direction from the fastening ring. By way of the longitudinal extension of the alignment portion there can be provided a rather precise longitudinal alignment of the alignment portion with the counter alignment structure as provided on or in the sidewall of the housing component. There can be provided a rather tilt free sliding motion of the alignment portion as the alignment portion slides along the complementary shaped counter alignment structure of the sidewall of the housing component during a longitudinal sliding insertion motion of the cartridge into the cartridge receiving space.

Moreover, the longitudinal extension may further serve as a handle or as a gripping feature to facilitate a mechanical handling of the fixing member, e.g. for attaching the same to the cartridge. In addition, the longitudinal extension of the alignment portion may protrude from only one longitudinal side or face of the fixing structure, thereby defining a single way of how to attach the fastening ring or the fastening portion to the cartridge. Generally, the fastening ring may be attachable to the neck of the cartridge in two different orientations. With the longitudinal extension as provided by the alignment portion there is provided a longitudinal symmetry breaking feature thus indicating to a user the only correct way of how to fix and arrange the fixing member to the cartridge.

Typically, the alignment portion should extend in proximal direction from the fastening portion when the fixing member is correctly assembled to the cartridge. In other words, the fixing element should protrude from the fastening portion towards the proximal end of the cartridge. Hence, it should face away or extend away from the distal outlet end of the cartridge when correctly attached or assembled to the cartridge. In case that a user should inadvertently attach the fastening portion to the neck of the cartridge in a wrong way the alignment portion, in particular the longitudinal extension thereof, may protrude in longitudinal direction from a distal end of the cartridge, thus preventing a complete insertion of the cartridge into the cartridge receiving space. Insofar, the longitudinal extension of the alignment portion also provides a twofold function. First of all, it serves to correctly align and to correctly orient the fixing structure of the fixing member relative to the counter fixing structure of the housing component. Second, the longitudinal extension provides a kind of a symmetry breaking feature, especially when the fixing structure comprises a fastening ring. The symmetry breaking feature defines the only correct way of how to attach the fixing member to the cartridge.

According to a further example the first fixing element comprises a snap element. The snap element comprises an abutment face facing towards a proximal longitudinal direction to abut with a complementary-shaped abutment face of the housing component. The first fixing element further comprises a beveled edge facing towards a distal longitudinal direction. Hence, the abutment face and the beveled edge or beveled flank are facing in opposite longitudinal directions.

This configuration of the first fixing element is particularly dedicated for a distally directed insert motion of the fixing member and the cartridge into the cartridge receiving space, e.g. via or through a proximally located insert opening of the housing component. The beveled edge of the fixing element serves to induce an elastic deformation of the fixing element and/or of the complementary shaped counter fixing structure of the housing component.

Upon reaching a final assembly configuration the abutment face of the fixing element gets in a longitudinal abutment with the complementary shaped abutment face of the housing component. The complementary shaped abutment face of the housing component typically faces in distal direction so as to engage with the proximally facing abutment face of the first fixing element. In this way, the fixing structure can be secured against a proximally directed removal of the fixing member out of the cartridge receiving space. Since the fastening portion of the fixing member remains permanently engaged with the neck portion also the entirety of the cartridge is effectively hindered to slide out of the cartridge receiving space in proximal direction.

With further examples also the second fixing element comprises a snap element, which may be somewhat identically shaped to the snap element as described above in connection with the first fixing element. In this way, there can be provided a twofold snap fastening acting in longitudinal direction as the cartridge provided with the fixing member reaches the final assembly position.

In another aspect there is provided a cartridge assembly for a drug delivery device. The cartridge assembly comprises a cartridge. The cartridge comprises a tubular shaped barrel containing a medicament. The cartridge, in particular its barrel comprises a neck portion, e.g. featuring a reduced diameter compared to the tubular shape of the main portion of the barrel. The cartridge assembly further comprises a housing component of a drug delivery device. The housing component is typically implemented as a cartridge holder. The housing component comprises a sidewall confining a cartridge receiving face extending in a longitudinal direction. Typically, the housing is also of a somewhat tubular shape. The housing component further comprises a counter fixing structure and a counter alignment structure. The counter fixing structure and/or the counter alignment structure is or are provided on or in the sidewall of the housing component.

The cartridge assembly further comprises a fixing member as described above. The fixing member is fastenable to the neck portion of the cartridge. When fastened to the neck portion of the cartridge the fixing member is configured to engage with a counter fixing structure and is further configured to align with the counter alignment structure before or in the course of inserting the cartridge into the cartridge receiving space.

Typically, and in the course of arranging or assembling the cartridge in the cartridge receiving space, the alignment portion of the fixing member attached to the cartridge is aligned with the counter alignment structure as provided on or in the sidewall of the housing component. A correct alignment of the alignment portion with the counter alignment structure allows and supports a longitudinal sliding insertion of the cartridge and the fixing member into the cartridge receiving face. Upon or when reaching a final assembly position, e.g. in which the cartridge receiving space is effectively occupied by the cartridge, the fixing structure of the fixing member engages with the counter fixing structure of the housing components thereby fixing and/or retaining the cartridge and the fixing member inside the cartridge receiving space.

Typically, and since the cartridge assembly makes use of a fixing member as described above all features, effects and benefits as described above in connection with the fixing member equally apply to the cartridge assembly comprising such a fixing member.

With some examples the fixing member is attachable or fixable to the neck portion of the cartridge, which is adjacent or close to a longitudinal distal end of the cartridge. The cartridge with the fixing member attached thereto is insertable in longitudinal distal direction through an insert opening of the housing component into the cartridge receiving space. Typically, the insert opening of the housing component for receiving the cartridge and the fixing member may be provided at or near a proximal end of the housing component. Since the fixing member is located at or near the distal end of the cartridge the counter fixing structure of the housing components to mechanically engage the fixing structure of the fixing member is also located at or near a distal end of the cartridge receiving space. Since the fixing structure is provided on one or several dedicated circumferential positions with regards to the tubular shape of the cartridge also the complementary shaped counter fixing structure of the housing component to engage with the fixing structure is or are provided only at well-defined dedicated circumferential or tangential positions.

In order to provide a proper mutual engagement between the fixing structure as provided on the fixing member and the counter fixing structure as provided e.g. on an inside of the sidewall of the housing component, there is provided the alignment portion on the fixing member, which is configured to engage and/or slide along the complementary shaped counter alignment structure on or in the sidewall of the housing component. The counter alignment structure may adjoin or may extend towards and into the proximal end face of the sidewall of the housing components. The counter alignment structure may enforce or require a correct alignment or orientation of the fixing member. Otherwise, insertion of the fixing member and the cartridge into the cartridge receiving space in distal direction may be blocked. Once the alignment portion of the fixing member is correctly aligned with the counter alignment structure as provided on or in the sidewall of the housing component a longitudinal sliding motion of the fixing member and the cartridge attached thereto into the cartridge receiving space is supported.

Here, the alignment portion and the counter alignment structure are in a kind of a keyed engagement inhibiting a rotation of the fixing member relative to the sidewall of the housing component while supporting a longitudinal non-rotating sliding displacement of the fixing member relative to the sidewall of the housing component. Here and during insertion of the cartridge with the fixing member attached thereto into the cartridge receiving space the engagement of the alignment portion with the counter alignment structure provides a kind of a non-rotating longitudinal sliding guide for the fixing structure to engage with the counter fixing structure on the inside of the housing component.

According to a further example the housing component comprises or forms a cartridge holder comprising a proximal end configured for attachment to a body of the drug delivery device. The proximal end of the cartridge holder further comprises an insert opening sized to receive the cartridge with the fixing member attached thereto in only one or several orientations of the cartridge and the fixing member relative to the cartridge holder. Here, an orientation of the cartridge or fixing member relative to the cartridge holder means a rotational position thereof with regard to the longitudinal direction of the housing component as an axis of rotation. Moreover, also a longitudinal symmetry axis of the tubular-shaped cartridge may serve as an imaginary axis of rotation with regards to which the cartridge and the fixing member attached thereto can be rotated in order to obtain a proper alignment with the counter alignment structure as provided on the sidewall of the cartridge holder.

The fixing member is typically provided as a separate piece. It may be mechanically fastened to the neck portion of the cartridge, simply by a kind of a mechanical clipping operation. Existing cartridges can be easily retrofitted with such fixing members. The fixing member comprises the fastening portion and a fixing structure, wherein the fixing structure is configured to engage with a complementary shaped counter fixing structure as provided on or in the housing component, hence on or in the cartridge holder. Since the position of the counter fixing structure may be difficult to identify from outside the cartridge holder the mutual alignment and guiding as provided by the alignment portion engaged with the counter alignment structure provides a respective longitudinal guiding for the fixing structure such that the fixing structure mechanically engages the counter fixing structure when the cartridge with the fixing member attached thereto reaches the final assembly configuration inside the cartridge receiving space.

According to a further example the counter alignment structure of the cartridge assembly comprises a longitudinally extending alignment recess on an inside of the sidewall of the cartridge holder. The longitudinally extending alignment recess is complementary shaped to the radially outwardly protruding alignment portion of the fixing member. This way, there can be provided a keyed engagement between the counter alignment structure of the sidewall and the alignment portion of the fixing member.

With another example it might be the counter alignment structure that comprises a radially inwardly protruding alignment portion to engage with a complementary shaped longitudinally extending alignment recess on an outside surface of the fixing member. In this way, there can be provided a likewise rotation inhibiting longitudinal guiding between the sidewall of the housing component and the fixing member attached to the cartridge.

According to a further example the counter fixing structure as provided in or on the sidewall is configured to engage with the first fixing element of the fixing member when the cartridge provided with the fixing member reaches a final assembly position inside the cartridge receiving space. Typically, the counter fixing structure and the first fixing element serve to engage in longitudinal or axial direction so as to axially or longitudinally retain the fixing member and hence the cartridge attached thereto inside the cartridge receiving space. Once engaged the fixing structure and the counter fixing structure prevent removal of the fixing member and hence of the cartridge from the cartridge receiving space towards the proximal direction.

The counter fixing structure and the fixing structure may engage in a snap like manner. Here, at least one of the fixing structure and the counter fixing structure may comprise a protrusion to engage with a complementary-shaped recess of the other one of the counter fixing structure and the fixing structure.

With some examples, the counter fixing structure of the cartridge holder may be void of any radially inwardly protruding fixing elements, that may otherwise harm the integrity or functionality of the dispensing end of the cartridge upon insertion of the cartridge into the cartridge receiving space. Here, it may be exclusively the fixing structure of the fixing member that comprises at least one or more radially outwardly extending fixing elements to engage with a or numerous radial recess(es) as provided on an inside of the sidewall of the cartridge holder.

According to a further example the first fixing element is longitudinally aligned with a first counter fixing element through the alignment of the alignment portion of the fixing member with the counter alignment structure of the sidewall upon insertion of the cartridge and the fixing member into the cartridge receiving space. The keyed engagement of the alignment portion with the counter alignment structure serves to guide and to longitudinally align the fixing structure with the counter fixing structure. The mutual engagement of the alignment portion with the counter alignment structure serves to prevent any rotation of the fixing member relative to the housing component during insertion into the cartridge receiving space. This way, and when correctly aligned the fixing structure and the counter fixing structure can be engaged by a distally directed sliding motion of the fixing member relative to the housing component or cartridge holder.

According to a further example the cartridge assembly may be implemented as a disposable cartridge assembly. Once the fixing structure engages the counter fixing structure the mutual engagement cannot be abrogated except through a destruction of e.g. the housing component. With other examples the cartridge assembly is implemented as a reusable cartridge assembly. Here, the counter fixing structure may allow and support a well-defined disengagement from the fixing structure. This may be provided e.g. by using a dedicated tool, particularly designed to modify the configuration or shape of the counter fixing structure, e.g. from outside the housing component. Here, and with some examples the counter fixing structure may comprise a recess in the sidewall of the housing component, which recess may be implemented as a through opening, thus allowing to urge the tool of appropriate size into and/or through the recess so as to bring the first fixing element out of engagement from the counter fixing structure. The same may also apply to configurations, wherein the fixing structure comprises a first fixing element and a second fixing element.

It may be then possible to remove the cartridge and the fixing member attached thereto from the cartridge receiving space in proximal direction. An empty cartridge may be then replaced by a new cartridge. The fixing member may remain attached to the cartridge or may be detached from the empty cartridge. It may be re-attached to the new cartridge. The fixing member and the new cartridge may be then inserted again into the cartridge receiving space of the housing component, e.g. implemented as cartridge holder.

According to another aspect the present disclosure also relates to a method of assembling a cartridge assembly of a drug delivery device, e.g. of an injection device, such as a pen-type injector. The method of assembling a cartridge assembly may be implemented or may be executed by making use of a cartridge assembly as described above. The method comprises the steps of fastening of a fixing member as described above to a neck portion of a cartridge, e.g. filled with a medicament. The method further comprises the step of aligning the alignment portion of the fixing member with a counter alignment structure as provided on or in a sidewall of a cartridge holder of a drug delivery device.

Thereafter, the method comprises the step of inserting the cartridge and the fixing member attached thereto into a cartridge receiving space of the cartridge holder. Typically, inserting the cartridge comprises a purely longitudinal sliding motion of the cartridge into the cartridge receiving space, wherein the mutual engagement of the alignment portion with the counter alignment structure serves to prevent a rotation of the cartridge or of the fixing member relative to the cartridge receiving space. Finally and in a further step the fixing structure of the fixing member is brought in engagement with the counter fixing structure when the cartridge reaches a final assembly position or during reaching such assembly position in the cartridge receiving space. Typically, engaging the fixing structure of the fixing member with the counter fixing structure provides and leads to a longitudinal or axial engagement and hence to a longitudinal fixing of the fixing member and hence of the cartridge to or inside the cartridge holder.

Typically and since the method of assembling a cartridge assembly can be conducted by making use of a cartridge assembly as described above and by making use of a fixing member as described above, all features, effects and benefits as described above in connection with the fixing member and the cartridge assembly equally apply to the method of assembling the cartridge assembly.

According to another aspect the disclosure also relates to a drug delivery device, e.g. implemented as an injection device, such as a pen type in injector. The drug delivery device comprises a body, e.g. in form of a proximal housing component. The body or the proximal housing component is typically connectable to a cartridge assembly as described above. The drug delivery device typically comprises a drive mechanism arranged inside the body and flesh or attached to the body, e.g. comprising a piston rod to exert a dispensing force onto a piston located inside the tubular shaped barrel of the cartridge, wherein the cartridge is typically provided and/or fixed inside the housing component or cartridge holder of the cartridge assembly. Typically, the cartridge assembly is configured to releasably or non-releasably connect and/or fix to the body of the drug delivery device.

The drug delivery device typically comprises a cartridge assembly as described above. Insofar, all features, effects and benefits as described above with regards to the cartridge assembly equally apply to the drug delivery device.

With some examples of the drug delivery device or of the cartridge assembly the fixing member may be mechanically coded to the cartridge or to the type of a cartridge. Hence, there may be selected one of a plurality of available and differently configured fixing members in accordance to the size of the cartridge and/or in accordance to the type of medicament contained in the cartridge.

Differently configured fixing members may distinguish from each other by at least one of a fastening portion, a fixing structure and an alignment portion. For instance, a first fixing member may be configured for engagement with only a first type of a cartridge. The first fixing member may comprise a first configuration of an alignment portion, which is exclusively suitable for insertion into a first housing component of a plurality of differently configured or available housing components. The first housing component may be mechanically coded to connect exclusively to only one of a number of available bodies or drive mechanisms of drug delivery devices. Accordingly, different encoded housing components or differently encoded cartridge holders may distinguish from each other by a shape and/or geometry of a cartridge receiving space, by the shape and/or configuration of a counter fixing structure and/or by a shape and/or configuration of a counter alignment structure.

In effect, a cartridge of a first type intended for exclusive use with a first cartridge holder, which cartridge holder is intended for exclusive use with a first body or drive mechanism of a drug delivery device, can be only and exclusively inserted and fastened to the cartridge holder by way of the fixing member. This way and with the fixing member attached to the cartridge of the first type insertion of this cartridge into any other cartridge holder, e.g. into a cartridge holder of a second type, can be effectively blocked and prevented. The cartridge holder of the second type may be exclusively dedicated for and may be exclusively connectable to a body or drug delivery device of a second type, which is dedicated to interact with a different cartridge, e.g. with a cartridge of a second type.

Cartridge assemblies comprising a cartridge, a cartridge holder and a fixing member, by way of which the cartridge is permanently fixed or is fixable inside the cartridge holder, can be manufactured and assembled by a pharmaceutical manufacturer. Such cartridges assemblies might be commercially distributed for reusable drug delivery devices. The cartridge holder of the cartridge assembly and the body of the drug delivery device may be mechanically encoded to prevent unintended cross use, i.e. to prevent use of a body and/or drive mechanism of a first type with a cartridge or cartridge holder of a second type, not intended for use with this particular body or drive mechanism. In effect and by providing cartridge assemblies with a cartridge fixed inside the drug delivery device patient safety can be enhanced and misuse of drug delivery devices can be effectively prevented.

Generally, the scope of the present disclosure is defined by the content of the claims. The injection device is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091 March-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1: 2014(E). As described in ISO 11608-1: 2014(E), needle-based injection systems may be broadly distinguished into multi-dose container systems and single-dose (with partial or full evacuation) container systems. The container may be a replaceable container or an integrated non-replaceable container.

As further described in ISO 11608-1: 2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1: 2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1: 2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

It will be further apparent to those skilled in the art that various modifications and variations can be made to the present disclosure without departing from the scope of the disclosure. Further, it is to be noted, that any reference numerals used in the appended claims are not to be construed as limiting the scope of the disclosure.

### BRIEF DESCRIPTION OF THE DRAWINGS

In the following, numerous examples of a data logging device for monitoring use of an injection device as well as a respective injection device will be described in greater detail by making reference to the drawings, in which:

FIG. 1 schematically shows an example of a drug delivery device,

FIG. 2 shows a cartridge provided with a fixing member,

FIG. 3 illustrates a cartridge assembly comprising a cartridge holder and a cartridge provided with a fixing member prior to insertion into the cartridge receiving space,

FIG. 4 is a perspective view of the isolated fixing member,

FIG. 5 is a side view of the fixing member of FIG. 4,

FIG. 6 is another side view of the fixing member,

FIG. 7 is a top view of the fixing member,

FIG. 8 is a perspective illustration of the housing component implemented as a cartridge holder,

FIG. 9 is a partially cut view through a distal portion of the cartridge assembly of FIG. 8,

FIG. 10 is a cross-section through the cartridge assembly of FIG. 9,

FIG. 11 is a longitudinal section through the cartridge assembly,

FIG. 12 is an enlarged illustration of the distal portion of the cartridge assembly of FIG. 11,

FIG. 13 is a flowchart of a method of assembly,

FIG. 14 is another longitudinal cross-section through the cartridge assembly,

FIG. 15 is an enlarged view of the distal portion of the illustration of FIG. 14, and

FIG. 16 is another illustration of the cross-section of FIG. 15.

### DETAILED DESCRIPTION

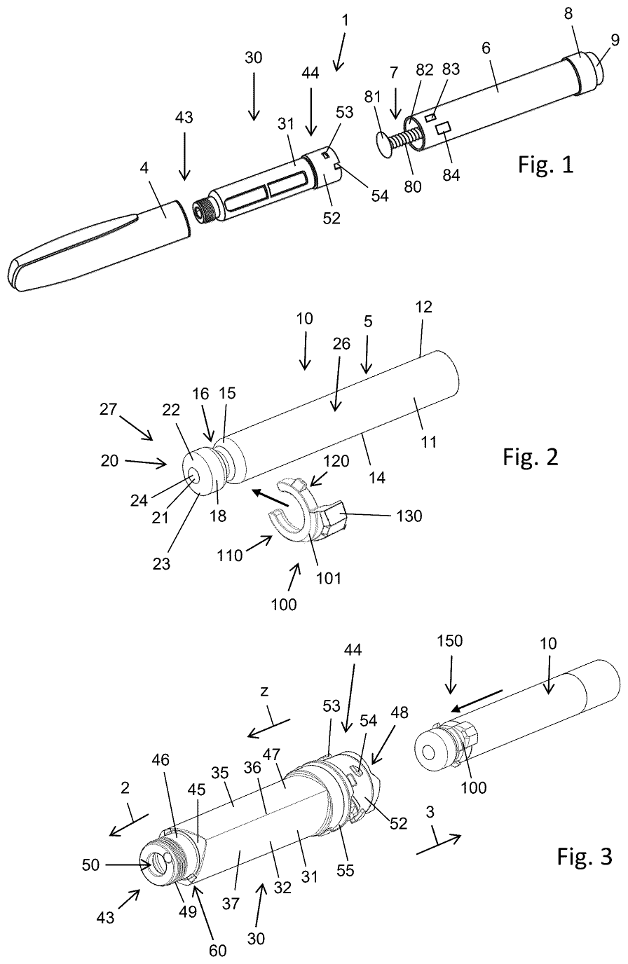

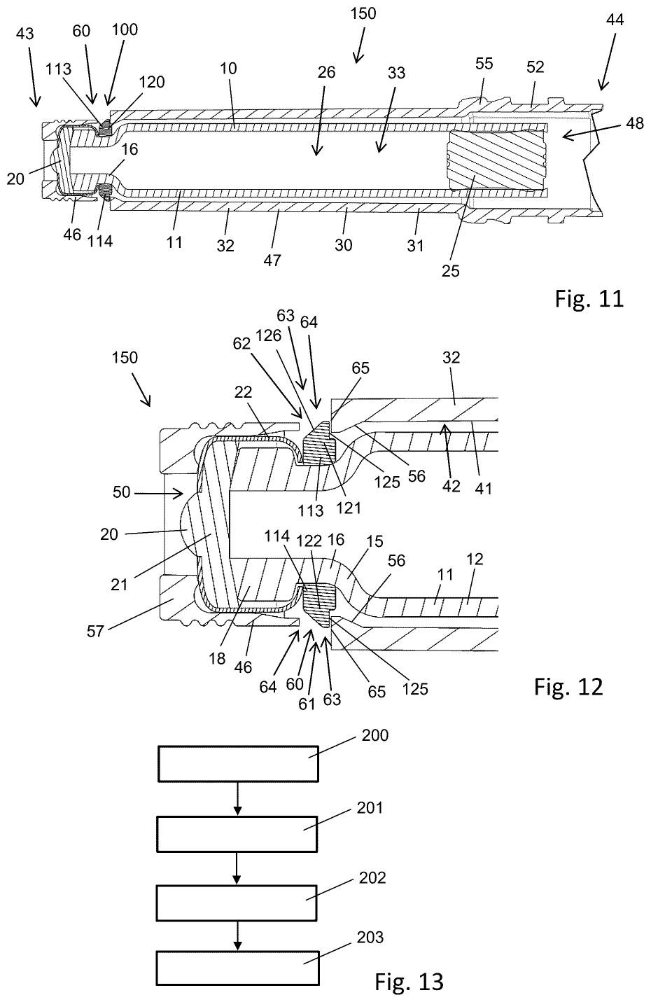

FIG. 1 shows an example of a drug delivery device 1 implemented as a pen-type injector. The drug delivery device 1 comprises numerous housing components. In detail the drug delivery device 1 comprises a tubular shaped housing component 30 implemented as a cartridge holder 31 sized to accommodate a medicament container 5, such as a cartridge 10, which is illustrated in FIG. 2. The drug delivery device 1 further comprises a body 6 forming or constituting a proximal housing component 6. The body 6 houses a drive mechanism 7. The drive mechanism 7 may comprise a dose member 8 and/or a trigger 9 for setting and/or dispensing a dose of a medicament 26 contained in a medicament container 5, which in the present case may be implemented as a cartridge 10. Typically, the drive mechanism 7 comprises a piston rod 80, e.g. provided with a pressure piece 81 at its distal end. The pressure piece 81 is configured to abut in longitudinal direction (z) with a proximal end face of a piston 25 slidably arranged inside the barrel 11 of the cartridge 10. The piston 25 is for instance illustrated in FIG. 11.

The barrel 11, which may be of a vitreous material, such as glass, comprises a sidewall 12 with a main portion 14 of substantially tubular shape. The piston 25 is slidably displaceable along the main portion 14. Towards the dispensing and 27 the main portion 14 merges into a narrow win shoulder portion 16, which in turn merges in to a radially stepped down neck portion 16. The neck portion 16 terminates with a head portion 18, which, at least on its outside surface may comprise a somewhat radially widened cross-section compared to the neck portion.

The drug delivery device 1 further comprises a protective cap 4 configured to cover the dispensing end 27 of the drug delivery device 1. Typically, the housing component 30 is implemented as a cartridge holder 31. The housing component 30 comprises a distal end 43 and a proximal end 44. The distal end 43 is configured to engage with a needle assembly, e.g. a double-tipped injection needle by way of which a dose of the liquid medicament 26 can be injected into biological tissue. For this, the distal end 43 of the cartridge holder 31 comprises a threaded section 49 which supports and allows for a threaded connection with a complementary shaped needle hub equipped with the injection needle.

The distal end 43 of the cartridge holder 31 further comprises an aperture 50 to receive a proximal portion or a proximally extending portion of the double-tipped injection needle. The injection needle is configured to pierce and/or to penetrate a seal 20 at the distal end of the cartridge 10. The seal 20 may comprise a pierceable septum 21, which is fixed to a distally located head portion 18 of the cartridge by way of a septum retainer 22. The septum retainer 22 may comprise a beaded cap 23, e.g. made of a pliable material, such as aluminum or plastic.

The septum retainer 22 comprises an aperture 24 that aligns with the aperture 50 when the cartridge 10 is received in the interior of the cartridge holder 31. Inside the cartridge holder 31 there is provided a cartridge receiving space 33, which is effectively occupied by the cartridge 10 when assembled inside the cartridge holder 31. The cartridge holder 31 comprises a proximal end 44, which may be provided with an insert 52 sized and configured for insertion into a receptacle 82 as provided at the distal end of the body 6. towards the distal direction 2, the insert 52 is delimited by a flange portion 55. When the cartridge holder 31 is attached to the body 6, the flange portion 55 may be in longitudinal abutment with a distal end face of the sidewall of the receptacle 82.

On the inside of the receptacle 82 there may be provided a counter fastening structure 83, e.g. in form of a radial protrusion or radial recess to engage with a complementary shaped fastening element 53 as provided on an outside surface of the insert 52. This way, the proximal end 44 of the cartridge holder 31 can be fixed and attached to the body 6.

Optionally, the proximal end 44 of the cartridge holder 31 comprises a coding element 54 by way of which the cartridge assembly 150 can be mechanically encoded to distinguish from another cartridge assembly 150, e.g. equipped with a different type of cartridge 10 or with a cartridge containing a different medicament 26. The coding element 54 or coding feature may be complementary shaped or complementary positioned to a counter coding element 84 as provided on or inside the body 6. A nonmatching pair of a coding element 54 and a counter coding element 84 may prevent a mutual fastening of the cartridge holder 31 to the body 6.

A mutual fastening and fixing of the cartridge holder 31 to the body 6 may be only provided and allowed when the coding element 54 of the cartridge holder 31 matches in size, shape and position with the corresponding counter coding element 84 as provided on or inside the body 6.

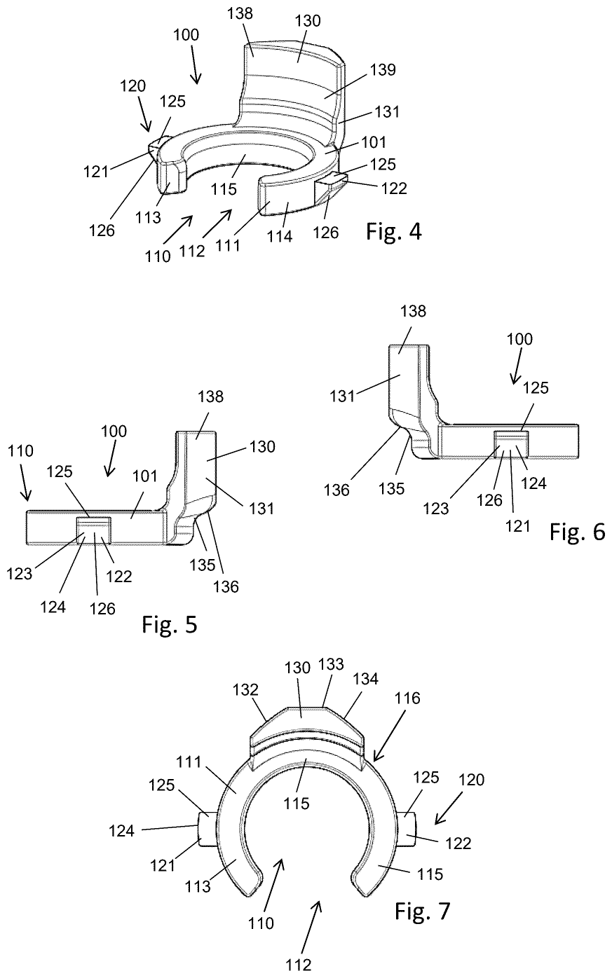

In one aspect the present disclosure relates to a cartridge assembly 150 comprising a cartridge holder 31, a cartridge 10 and a fixing member 100. The fixing member 100, which is illustrated in greater detail in FIGS. 4-7 comprises a body 101. The fixing member 100, in particular its body 101 may be a single pieced or unitary component. The fixing member 100 is provided separate from the cartridge 10. It can be fastened to the cartridge 10, in particular to a neck portion 16 of the barrel 11 of the cartridge 10. For fixing the fixing member 100 to the cartridge 10 the fixing member comprises a fastening portion 110. The fastening portion 110 is configured to fix the entire fixing member 100 to the neck portion 16 of the cartridge 10.

With the example as illustrated in FIGS. 4-7 the fastening portion 110 comprises a fastening ring 111. The fastening ring 111 is of substantially annular shape. It is interrupted by a slit opening 112, thereby dividing the fastening ring into a first ring portion 113 and a second ring portion 114. The first ring portion 113 and the second ring portion 114 are interconnected the a middle portion 115. The first ring portion 113, the middle portion 115 and the second ring portion 114 may comprise an incomplete annular shape extending in the tangential or circumferential direction from about 210° to 300°. In the presently illustrated example the tangential extension of the fastening ring 111 is about 270°. The size of the slit opening 112 is slightly smaller than the diameter of the neck portion 60 of the cartridge 10. The fastening ring 111 may be pliable or elastically deformable to a certain extent so as to enable a clip fastening onto the neck portion 16 of the cartridge 10.

The fixing member 100 further comprises a fixing structure 120. The fixing structure 120 is configured to fix the fixing member 100 inside the cartridge receiving space 33 as illustrated in FIG. 11. For this, the fixing structure 120 comprises at least a first fixing element 121, which is configured to engage with a counter fixing structure 60 of the housing component 30, hence of the cartridge holder 31. In addition, the fixing member 100 comprises an alignment portion 130. The alignment portion 130 is configured to align with a counter alignment structure 34, which is provided on or in the sidewall 32 of the cartridge holder 31.

As it becomes apparent from the illustration of the numerous FIGS. 1-16 the fixing member 100 is configured for fastening to a distal portion of the cartridge 10. It is then configured for insertion into and through an insert opening 48 of the cartridge holder 31, which insert opening 48 is provided at or near the proximal end 44 of the cartridge holder 31. Consequently, and when the cartridge 10 reaches a final assembly position, as e.g. illustrated in FIG. 11, the fixing member 100 is located at or near the distal end 43 of the cartridge holder 31. There, the cartridge holder 31 comprises a counter fixing structure 60, to engage with the fixing structure 120 as provided on the fixing member 100.

Since the fixing member 100 is configured for insertion into the cartridge receiving space 33 obtaining of a proper alignment of the fixing structure 120 with the counter fixing structure 60 may be difficult to achieve. For this, the alignment portion 130 is configured to engage with the counter alignment structure 34 as provided on the inside 41 of the sidewall 32 of the cartridge holder 31. The alignment portion 130, which is positionally fixed relative to the fixing structure 120 can be and remain in a keyed engagement with the sidewall 32 of the cartridge holder 31 during and for insertion of the cartridge 10 into the cartridge holder 31. The keyed engagement between the alignment portion 130 and the counter alignment structure 34 rotationally locks the fixing member 100 relative to the cartridge holder 31 and longitudinally guides the fixing structure 120 into engagement with the counter fixing structure 60 of the cartridge holder 31.

As illustrated in greater detail in FIGS. 4-7 the alignment portion 130 comprises a radial protrusion 131 protruding radially outwardly from an outside surface 116 of the fastening portion 110. In this way the alignment portion 130 provides a symmetry breaking of the fastening portion 110 and hence of the fastening ring 111. As shown in FIG. 4 the alignment portion 130 protrudes in longitudinal direction from the plane of the fastening ring 111. As particularly illustrated in FIG. 2, the alignment portion 130 protrudes towards the proximal end of the cartridge 10 when the fixing member 100 is fastened and attached to the neck portion 16. This way, fixing elements 121, 122 as provided on an outside surface of the fastening portion 110, in particular as provided on an outside surface of the first and second ring portions 113, 114 are oriented in a correct way.

The alignment portion 130 further comprises a longitudinal extension 138 protruding in longitudinal direction from the fastening portion 110. The longitudinal extension 138 provides a symmetry breaking feature for the fastening ring 111. When attached to the cartridge the longitudinal extension 138 it should protrude in proximal direction 3 from the fastening portion. In this way, the fixing structure 120 will be correctly oriented with regard to the longitudinal direction (z). Moreover, the longitudinal extension 138 may also serve as a handle so as to facilitate gripping and/or fastening the fixing member 100 to the cartridge 10.

As particularly illustrated in FIG. 4-7 the fixing structure 120 comprises a first fixing element 121 provided on the first ring portion 113. The fixing structure 120 further comprises a second fixing element 122 provided on the second ring portion 114. The fixing elements 121, 122 are implemented as a snap element 123. They comprise a radially outwardly protruding nose 124 or nose portion with an abutment face 125 facing in proximal direction 3, hence facing towards the alignment portion 130. In the opposite axial direction, i.e. towards the distal direction 2, the fixing elements 121, 122, each comprise a beveled edge 126 or beveled flank. The beveled edge extends radially outwardly and merges into the radially stepped down abutment face 125.

On an outside facing portion of the fixing member 100 the alignment portion 130 merges into the fastening portion 110 via a shoulder portion 135. The shoulder portion 135 comprises a stepped profile and a distally facing abutment face 136 by way of which the fixing member 100 adapts the shape of the inside surface of the shoulder portion 45 of the sidewall 32 of the cartridge holder 31. As illustrated in FIG. 15 the cartridge holder 31 comprises a sidewall 32 comprising a tubular-shaped main portion 47, which converges into a radially stepped down neck portion 46, which is provided with the threaded section 49. The tubular or main portion 41 of the sidewall 32 radially narrows via the shoulder portion 45 into the neck portion 46.

As shown in greater detail in FIG. 15 an outside facing shape of the shoulder portion 135 of the fixing member 100 is complementary shaped to the inside shape of the shoulder portion 45 of the sidewall 32 of the cartridge holder 31.

As further illustrated in FIG. 15 an inside surface 139 of the extension 138 of the alignment portion 130 is shaped and configured to abut with the shoulder portion 15 of the barrel 11 of the cartridge 10. In this way and when reaching a final assembly position the fixing member 100 does not only provide a longitudinal fixing of the cartridge 10 inside the cartridge holder 31 but also provides a well-defined axial or longitudinal abutment between the outside facing shoulder portion 15 of the cartridge 10 and an inside facing shoulder portion 45 of the cartridge holder 31.

As it is particularly illustrated in FIG. 7 the alignment portion 130 protrudes radially outwardly from the annular shape of the fastening portion 110. The alignment portion 130, in particular the entirety of the longitudinal extension 138 comprises a beveled outside surface 132, another beveled outside surface 134 and a further outside surface 133 located between the two beveled outside surfaces 132, 134. In the cross-section of FIG. 7 the outside surfaces 132, 133, 134 are of trapezoidal shape or form a trapezoidal profile of the extension 138. Here, and generally, there may be available also some other geometric profiles enabling a mechanical coding of the fixing member 100 and the cartridge holder 31.

On the inside of the sidewall 32 of the cartridge holder 31 there is provided at least one alignment recess 42 that is complementary shaped to the cross-section of the alignment portion 130. The alignment portion 130 in alignment with the alignment recess 42 is particularly illustrated in FIG. 10. In FIG. 10 there is illustrated a specific example of a cartridge holder 31, wherein the sidewall 32 slightly deviates from a cylindrical cross sectional shape. Here, the sidewall 32 comprises a first sidewall section 35, a second sidewall section 37 and a third side wall section 39. Each side wall section 35 comprises a convex shaped panel or slat profile as seen from outside. Each side wall section 35, 37, 39 may comprise a continues profile extending in longitudinal direction. The first sidewall section 35 merges into the second sidewall section 37 along a ridge portion 36. The second sidewall section 37 merges into the third sidewall section 39 along a second ridge portion 38 and the third side wall section 39 merges into the first sidewall section 35 along a third ridge portion 40.

In the cross-section of FIG. 10 there are illustrated two further alignment recesses 42′, 42″ that are located radially inwardly from the second and third ridge portions 38, 40. The alignment recesses 42 may be likewise suitable to receive the alignment portion 130. Hence, the sidewall 32 of the cartridge holder 31 may provide and support three different alignment configurations of the fixing member 100 relative to the holder 31. The different alignment configurations may transform into each other by a rotation of about 120° with the central axis of the cartridge 10 as an imaginary axis of rotation.

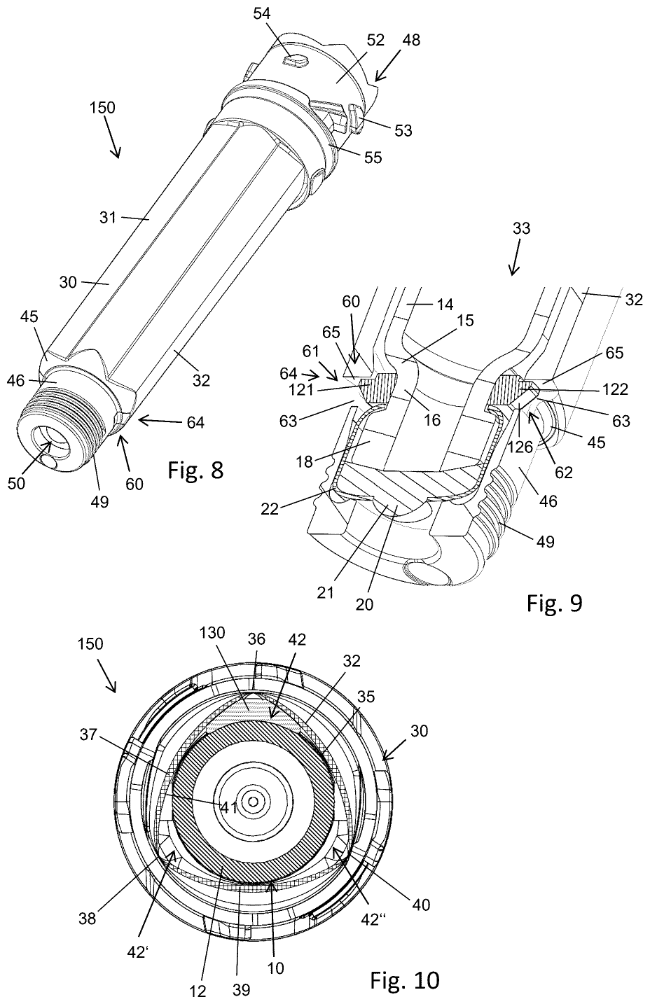

As it is further apparent from FIGS. 8 and 9 the counter fixing structure 60 comprises at least two counter fixing elements 61, 62, which in the present embodiment are configured as recesses 63 in the sidewall 32 of the cartridge holder 31. The counter fixing elements 61, 62 are provided as recesses 63 on the inside surface 41 of the sidewall 32. They are implemented as a through openings 64 as illustrated in FIG. 8. The counter fixing elements 61, 62 may be located at geometrically oppositely located positions so as to engage with the radially outwardly protruding fixing elements 121, 122 of the fixing structure 120 of the fixing member 110. In accordance to the number of available alignment recesses 42, 42′, 42″ on or along the inside surface 41 of the sidewall 32 there may be provided a respective number of counter fixing element 61, 62.

With further examples, wherein there is provided only a first and a second counter fixing element 61, 62 there may be provided only one alignment recess 42 on the inside 41 of the sidewall 32 thus enforcing only one dedicated alignment or orientation of the fixing member 100 relative to the internal keying or coding structure of the sidewall 32 of the cartridge holder 31.

The alignment recess 42 may extend all along the main portion 47 of the sidewall 32 of the cartridge holder 31. It may adjoin the insert opening 48 through which the fixing member 100 attached to the cartridge 10 is to be inserted in a longitudinal distal direction 2 into the cartridge receiving space 33. While the alignment portion 130 is and remains in engagement with the counter alignment structure 34, e.g. with the alignment recess 42, there is provided a keyed and rotation inhibiting engagement between the fixing member 100 and the cartridge holder 31.

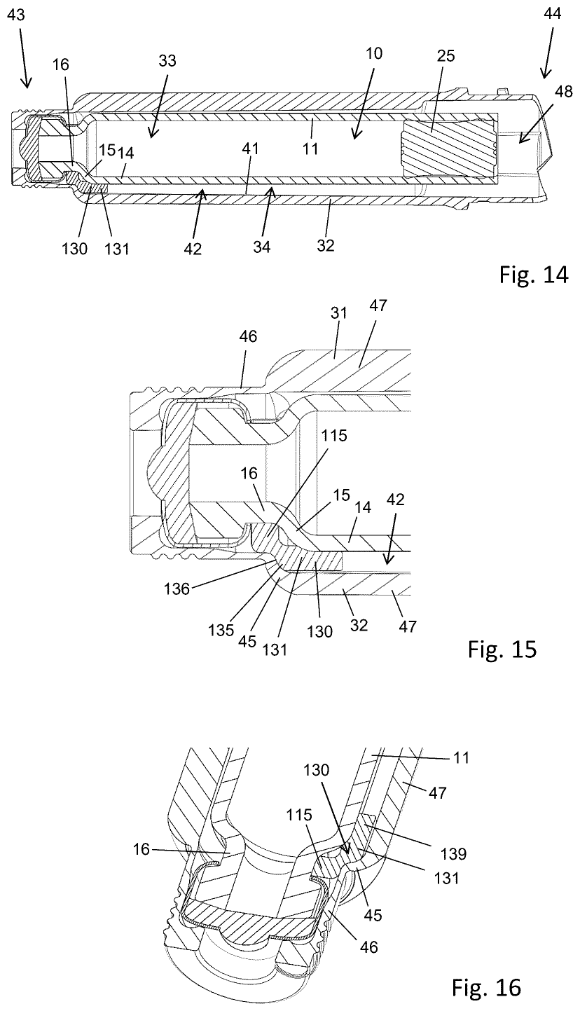

As the cartridge 10 and the fixing member 100 are urged in distal direction 2 towards the final assembly position as illustrated in FIGS. 11 and 14 the distally facing beveled edges 126 of the fixing elements 121, 122 may start to slide along a complementary shaped beveled edge 56 protruding radially inwardly from the inside surface 41 of the sidewall 32 at a position longitudinally adjacent to the position of the counter fixing elements 61, 62. In this way, the sidewall 32 may experience a slightly outwardly directed deformation until the beveled edge 126 or beveled flank of the fixing elements 121, 122 have passed the inwardly protruding beveled edge 56 or beveled flank.

Once the beveled edges 126 have longitudinally passed the beveled edges 56 the proximally facing abutment face 125 of the fixing elements 121, 122 get in longitudinal abutment with the distally facing abutment faces 65 of the counter fixing element 61, 62, thereby inhibiting a proximally directed displacement and hence a removing of the fixing member 100 in proximal direction 3 out of the cartridge receiving space 33.

A user trying to pull the cartridge 10 in proximal direction 3 out of the cartridge holder 31 will induce a proximally directed force effect of the head portion 18 being in abutment with the distally facing side of the fastening ring 111. Accordingly, a respective proximal force effect will be transferred to the fastening portion 110, which force is counteracted by the distally facing abutment faces 65 of the counter fixing structure 60.

With some examples of the cartridge assembly 150, wherein the counter fixing structure 60 comprises one or numerous through openings 64 being accessible from outside, it is generally conceivable to induce a deformation of at least one of the fixing element 121, 122 and the counter fixing structure 60, e.g. by a dedicated tool, so as to abrogate the axial or longitudinal fixing abutment between the abutment face 65 and the abutment face 125.

Towards a distal end of the cartridge 10, e.g. a distal end face of the septum retainer 22 may be in abutment with an end wall 57 of the cartridge holder 31. Furthermore, and as described above in connection with FIG. 15 the fixing member 100 may provide a respective distally acting abutment between the distally facing shoulder portion 15 of the barrel 11 of the cartridge 10 and the proximally facing shoulder portion 135 of the cartridge holder 31.

In the flowchart of FIG. 13 a method of assembling a cartridge assembly 150 as described herein is further described. In a first step 200 the fixing member 100 is fastened to the neck portion of the cartridge 10. Here, the fastening ring 111 may be clipped onto the radially narrowed neck portion 16. Thereafter, in step 201 the assembly of the fixing member attached to the cartridge is aligned and oriented in such a way that the alignment portion 130 the fixing member 100 is in alignment with the counter alignment structure 34 as provided on or inside the sidewall 32 of the cartridge holder 31.

With a correct alignment, e.g. when the radial protrusion 131 of the alignment portion 130 is oriented and aligned with the longitudinally extending alignment recess 42 as provided on the inside surface 41 of the sidewall 32 the cartridge 10 provided with the fixing member 100 can be inserted into the cartridge receiving space 33 in longitudinal direction towards the distal end 43 of the cartridge holder 31 in step 202.

Thereafter and upon or before reaching the final assembly position the fixing structure 120 of the fixing member 100 is brought in engagement with the counter fixing structure 60 in step 203. Here and as illustrated in FIG. 12 the radially outwardly extending protruding snap element 123 of at least one of the first and the second fixing elements 121, 122 mechanically engages with the recess 63 of the counter fixing element 61, 62, thereby inhibiting and blocking a proximally directed removal of the cartridge 10 from the cartridge receiving space 33.

### REFERENCE NUMBERS

1 drug delivery device
2 distal direction
3 proximal direction
4 protective cap
5 medicament container
6 body
7 drive mechanism
8 dose member
9 trigger
10 cartridge
11 barrel
12 sidewall
14 main portion
15 shoulder portion
16 neck portion
18 head portion
20 seal
21 septum
22 septum retainer
23 beaded cap
24 aperture
25 piston
26 medicament
27 dispensing end
30 housing component
31 cartridge holder
32 sidewall
33 cartridge receiving space
34 counter alignment structure
35 sidewall section
36 ridge portion
37 sidewall section
38 ridge portion
39 sidewall section
40 ridge portion
41 inside surface
42 alignment recess
43 distal end
44 proximal end
45 shoulder portion
46 neck portion
47 main portion
48 insert opening
49 threaded section
50 aperture
52 insert
53 fastening element
54 coding element
55 flange portion
56 beveled edge
57 end wall
60 counter fixing structure
61 counter fixing element
62 counter fixing element
63 recess
64 through opening
65 abutment face
80 piston rod
81 pressure piece
82 receptacle
83 counter fastening structure
84 counter coding element
100 fixing member
101 body
110 fastening portion
111 fastening ring
112 slit opening
113 ring portion
114 ring portion
115 middle portion
116 outside surface
120 fixing structure
121 fixing element
122 fixing element
123 snap element
124 nose
125 abutment face
126 beveled edge
130 alignment portion
131 protrusion
132 outside surface
133 outside surface
134 outside surface
135 shoulder portion
136 abutment face
138 extension
139 inside surface
150 cartridge assembly

1. A fixing member for fastening to a cartridge, wherein the cartridge is configured for insertion into a housing component of a drug delivery device, wherein the housing component comprises a sidewall confining a cartridge receiving space, the cartridge receiving space extending in a longitudinal direction, the fixing member comprising:
a fastening portion for fastening to a neck portion of the cartridge,
a fixing structure comprising a first fixing element configured to engage with a counter fixing structure of the housing component, and
an alignment portion configured to align with a counter alignment structure provided on or in the sidewall of the housing component.

2. The fixing member according to claim 1, wherein the alignment portion is configured to orient the fixing member in at least one correct or dedicated orientation relative to the housing component and/or relative to the counter fixing structure.

3. The fixing member according to claim 1, wherein the counter alignment structure extends along the longitudinal direction and wherein the alignment portion is configured to engage and/or to slide along the counter alignment structure.

4. The fixing member according to claim 1, wherein when fastened to the neck portion of the cartridge inserting of the fixing member into the housing component is only possible with the alignment portion correctly align and correctly oriented with regard to the counter alignment structure.

5. The fixing member according to claim 1, wherein when the alignment portion is misaligned relative to the counter alignment structure the fixing structure will not be able to engage the counter fixing structure.

6. The fixing member according to claim 1, wherein the fixing structure is configured to engage with the counter fixing structure in longitudinal direction and to fix the cartridge relative to the housing component with regard to the longitudinal direction.

7. The fixing member according to claim 1, wherein the alignment portion comprises one of a radial protrusion and a radial alignment recess to engage with one of a complementary shaped radial alignment recess and a radial protrusion on an inside surface of the sidewall of the housing component.

8. The fixing member according to claim 1, wherein the fastening portion comprises a fastening ring comprising a first ring portion and a second ring portion separated from each other by a slit opening.

9. The fixing member according to claim 8, wherein the fastening ring comprises a middle portion interconnecting the first ring portion and the second ring portion.

10. The fixing member according to claim 9, wherein the alignment portion is provided on an outside surface of the middle portion.

11. The fixing member according to claim 8, wherein the fixing structure further comprises a second fixing element, wherein the first fixing element is provided on the first ring portion and wherein the second fixing element is provided on the second ring portion.

12. The fixing member according to claim 8, wherein the alignment portion comprises a longitudinal extension protruding in longitudinal direction from the fastening ring.

13. The fixing member according to claim 1, wherein the first fixing element comprises a snap element, the snap element comprises an abutment face facing towards a proximal longitudinal direction to abut with a complementary shaped abutment face of the housing component and further comprises a beveled edge facing towards a distal longitudinal direction.

14. A cartridge assembly for a drug delivery device, comprising:
a cartridge comprising a tubular shaped barrel containing a medicament, wherein the cartridge comprises a neck portion,
a housing component of a drug delivery device, the housing component comprising:
a sidewall confining a cartridge receiving space extending in a longitudinal direction,
a counter fixing structure,
a counter alignment structure provided on or in the sidewall,
a fixing member according to claim 1 fastenable to the neck portion, and, when fastened to the neck portion of the cartridge being configured to engage with the counter fixing structure and to align with the counter alignment structure upon insertion of the cartridge into the cartridge receiving space.

15. The cartridge assembly according to claim 14, wherein the housing component comprises a cartridge holder comprising a proximal end configured for attachment to a body of the drug delivery device, wherein the proximal end comprises an insert opening sized to receive the cartridge with the fixing member attached thereto in only one or several orientations of the cartridge and the fixing member relative to the cartridge holder with regard to the longitudinal direction as an axis of rotation.

16. The cartridge assembly according to claim 14, wherein the counter alignment structure comprises a longitudinally extending alignment recess on an inside of the sidewall, which longitudinally extending alignment recess is complementary shaped to the radially outwardly protruding alignment portion of the fixing member.

17. The cartridge assembly according to claim 14, wherein the counter fixing structure provided in or on the sidewall is configured to engage with the first fixing element of the fixing member when the cartridge provided with the fixing member reaches a final assembly position inside the cartridge receiving space.

18. The cartridge assembly according to claim 17, wherein the first fixing element is longitudinally aligned with a first counter fixing element through an alignment of the alignment portion of the fixing member with the counter alignment structure of the sidewall upon insertion of the cartridge and the fixing member into the cartridge receiving space.

19. A method of assembling a cartridge assembly of a drug delivery device, the method comprising the steps of:
fastening of a fixing member according to claim 1 to a neck portion of a cartridge,
aligning the alignment portion with a counter alignment structure provided on or in a sidewall of a cartridge holder of the drug delivery device,
inserting the cartridge and the fixing member into a cartridge receiving space of the cartridge holder, and
bringing the fixing structure of the fixing member in engagement with the counter fixing structure when the cartridge reaches a final assembly position in the cartridge receiving space.

20. A drug delivery device, comprising:
a body,
a drive mechanism arranged inside or attached to the body and comprising a piston rod configured to exert a dispensing force onto a piston located inside a barrel of a cartridge,
a cartridge assembly according to claim 14 and connectable to the body.
